(19) United States
(12) Patent Application Publication
(10) Pub. No.: US 2010/0281585 A1
(43) Pub. Date: Nov. 4, 2010

(54) COMPOSITION AND METHOD FOR INCREASING RESISTANCE AGAINST PLANT PATHOGEN BY COMPRISING BACTERIAL GENETIC MATERIALS, AND PLANT PRODUCED BY THE METHOD

(75) Inventors: Choong Min Ryu, Daejeon (KR); Bo Young Lee, Chungcheongbuk-do (KR); Soo Hyun Lee, Daejeon (KR)

Correspondence Address: OSHA LIANG L.L.P., TWO HOUSTON CENTER, 909 FANNIN, SUITE 3500, HOUSTON, TX, 77010, US

(73) Assignee: KOREA RESEARCH INSTITUTE OF BIOSCIENCE AND BIOTECHNOLOGY, Yuseong-gum Daejeon (KR)

(21) Appl. No.: 12/744,008

(22) PCT Filed: Nov. 23, 2007
(86) PCT No.: PCT/KR07/05952

§ 371 Date: Jul. 8, 2010

(30) Foreign Application Priority Data
Nov. 20, 2007 (KR) 10-2007-0118267

(51) Int. Cl. A01H 5/00; C07H 21/02; A61K 31/7088

(52) U.S. Cl. 800/301; 536/23.1; 514/44 R

(57) ABSTRACT

The present invention relates to a composition for increasing resistance to plant pathogen by inducing an immune reaction of a plant wherein said composition comprises bacterial genetic materials as an effective component, a method for increasing resistance to plant pathogen by inducing an immune reaction of a plant wherein said method comprises a step of treating the plant with bacterial genetic materials, a plant produced by mentioned method to have increased resistance to plant pathogen, and seeds of such plant.

### TECHNICAL FIELD

The present invention relates to a composition for increasing resistance against plant pathogen by inducing an immune reaction of a plant wherein said composition comprises bacterial genetic materials as an inducing/effective component, a method for increasing resistance to plant pathogen by inducing an immune reaction of a plant wherein said method comprises a step of treating the plant with bacterial genetic materials, a plant produced by said method to have increased resistance to plant pathogen, and seeds of such plant.

### BACKGROUND ART

Various mechanisms have been developed by plants to protect themselves from many different microorganisms. Among the reactions by plants in response to such microorganisms, a reaction to pathogenic microorganisms (i.e., pathogens) is important in that it is a mechanism directly related to yield loss of crops. As such, many researches have been studied thereon. Especially, a current progress in a field of molecular biology provides much new information which has been unknown before. When a pathogen attacks a plant, the pathogen is first confronted with a cell wall of the plant. Responding to such attack, the plant has to recognize the microorganism and then express proper resistance to it to guarantee its own survival. In contrast to an immune reaction by an animal, such resistance presented by plant is called “plant innate immunity” or “basal resistance”. Factors that are common in such microorganisms to cause an expression of innate immunity in host are called microbe-associated molecular pattern (MAMP). Studies about MAMP were first reported by a German group led by G. Felix and T. Boller. While studying flagellins from various bacteria, they found that plants can recognize the protein, flagellin, and further confirmed that N-terminal 22-mer fragment of bacterial flagella plays a key role for such reaction. They named the 22-mer fragment ‘flg22’ (Zifpel et al., 2004, Nature 428:764-767). In addition, plant receptors that can recognize flg22 were found in Arabidopsis thaliana. Consequently, the receptor was identified as a transmembrane receptor-kinase (LRR-receptor-like kinase) having an extracellular LRR domain and was named ‘FLS2.’ The receptor was found to have many characteristics that are similar to TOLL receptor, a system of fruit fly Drosophila for recognizing bacteria, evidencing the presence of such system not only in an animal but also in a plant. PAMP, which is common to both animal and plant, includes many varieties in addition to fls22 and many studies are now actively being carried out therefor.

Meanwhile, although there has been a study reporting that bacterial genetic materials (i.e., DNA or RNA, etc.) work as MAMP in an animal system, no such result was presented for a plant.

According to Korean Patent Application Laid-Open No. 2003-0031485, a method for preventing and treating an infectious disease or other diseases by using monosaccharides or disaccharides is disclosed. However, it is different from MAMP used in the present invention.

### DETAILED DESCRIPTION OF THE INVENTION

### Technical Goal of the Invention

The present invention aims to develop a completely new agent for protecting plant, by proving that bacterial genetic materials can function as MAMP which induces innate immunity as a basic means for a plant to resist pathogen(s).

### Disclosure of the Invention

In order to achieve the technical subject as described above, the present invention provides a composition for increasing resistance to plant pathogen by inducing an immune reaction of a plant wherein said composition comprises bacterial genetic materials as an effective component.

Furthermore, the present invention provides a method for increasing resistance to plant pathogen by inducing an immune reaction of a plant wherein said method comprises a step of treating the plant with bacterial genetic materials.

Still furthermore, the present invention provides a plant produced by said method to have increased resistance to plant pathogen and seeds of such plant.

### EFFECT OF THE INVENTION

Based on the fact that genetic materials of bacteria, especially bacterial RNA, can induce innate immunity in plant, it is expected from the present invention that a technology relating to environmentally friendly pesticides is successfully developed.

### BRIEF DESCRIPTION OF THE DRAWINGS

FIG. 1 shows resistance to a plant pathogen wherein the resistance of a plant was induced by treating the plant with bacterial RNA; A) effect of alleviating a symptom of disease by treating the plant with RNA from Pseudomonas syringae pv. tomato strain DC3000, B) effect of alleviating a symptom of disease by treating the plant with RNA from Pseudomonas syringae pv. tomato strain DC3000, a gram-negative bacteria, and C) effect of alleviating a symptom of disease by treating the plant with RNA from Paenibacillus polymyxa strain E681, a gram-positive bacteria.

FIG. 2 shows a gene expression profile from microarray analysis and an analysis of gene ontology of the plant, which has been treated with bacterial RNA.

### BEST MODE FOR CARRYING OUT THE INVENTION

In order to achieve the technical subject as described above, the present invention provides a composition for increasing resistance to plant pathogen by inducing an immune reaction of a plant wherein said composition comprises bacterial genetic materials as an effective component. Preferably, mentioned bacterial genetic material is a bacterial RNA.

Mentioned bacteria include the following gram-negative bacteria; Haemophilus influenzae, Yersinia pestis, Escherichia coli, Salmonella typhi, Salmonella typhimurium, Vibrio cholerae, Pseudomonas aeruginosa, and Pseudomonas syringae, but are not limited thereto, as well as the following gram-positive bacteria; genus Bacillus consisting of Bacillus stearothermophilus, Bacillus cereus, and Bacillus anthrasis, Paenibacillus polymyxa, Clavibacter michiganensis, and Pectobacterium carotovorum, but are not limited thereto.

The present invention is the first report indicating that bacterial genetic materials can function as PAMP (pathogen associated molecular pattern) or MAMP (microbe-associated molecular pattern) which can induce innate immunity in plant. When a plant is treated with bacterial genetic materials, resistance of plant to pathogens is increased so that a development of an agent for protecting plants becomes possible by using stabilized bacterial genetic materials.

According to a composition of one embodiment of the present invention, it can further comprise inducing chemicals such as BTH (2,1,3-benzothiadiazole), ethephon, salicylic acid, methyl jasmonate, or DL-β-amino-n-butyric acid. It is known that said substances may induce disease resistance in plant. In addition to said substances, any substance that is publicly known in the pertinent art to induce resistance to plant pathogens by inducing an immunological reaction in plant can be further comprised.

According to a composition of one embodiment of the present invention, it can further comprise a buffer agent, carrier, auxiliary agent or vehicle that are agro-pharmaceutically allowed and well known in the pertinent art. The composition of the present invention can be lyophilized by lyophilization (i.e., freeze-dry), spray drying or spray cooling.

It is intended in the present specification that the term “buffer agent” means an aqueous solution comprising a mixture of acid and base to stabilize pH of the composition. As a buffer agent, TRIS, phosphate, carbonate, acetate, citrate, glycolate, lactate, borate, tartrate, carcodylate, ethanolamine, glycine, imidazole, and imidazole lactic acid, etc. can be used.

It is intended in the present specification that the term “diluting agent (or carrier)” means an aqueous or non-aqueous solution which is used for the purpose of diluting genetic materials of bacteria. The diluting agent can be at least one of saline, water, polyethylene glycol, propylene glycol, ethanol or oil (e.g., corn oil, peanut oil, cottonseed oil or sesame oil).

It is intended in the present specification that the term “auxiliary agent” means a certain chemical compound which is added to a preparation in order to increase a biological effect of bacterial genetic materials.

A vehicle can be one or more of carbohydrate, polymer, lipid and inorganic substance. Examples of carbohydrate include lactose, sucrose, mannitol and cyclodextrin that are added to a composition to facilitate lyophilization.

Examples of polymer include starch, cellulose ether, cellulose carboxylmethylcellulose, alginate, carrageenan, hyaluronic aicd, polyacrylic acid, polysulfonate, polyethyleneglycol/polyethylene oxide, polyvinyl alcohol/polyvinylacetate having different degree of hydrolysis and polyvinyl pyrrolidone (includes everything with a different molecular weight).

The composition of the present invention can be prepared as a formulation which includes emulsion, oil, hydrate, powder, granule, tablet, aerosol, suspension, and ointment, etc. If necessary, an emulsifying agent, a suspending agent, a spreading agent, a penetrating agent, a wetting agent, a thickening agent (muscilage, etc.) and a stabilizer, etc. can be further incorporated. Said formulation can be prepared in accordance with a method that is publicly known in the pertinent art.

According to a composition of one embodiment of the present invention, the above-mentioned plant pathogen can be selected from a group consisting of gram-positive bacteria, gram-negative bacteria and fungi. Preferably, mentioned plant pathogen can be Clavibacter michiganensis subsp. Michiganensis, Pseudomonas syringae pv. tomato DC3000, Pectobacterium carotovorum subsp. Carotovorum, Xanthomonas campestris pv. vesicatoria 833, Xanthomonas campestris pv. vesicatoria 833 pila, Pectobacterium carotovorum subsp. Atrosepticum, Acidovorax konjaci, Xanthomonas albilineans, Xanthomonas oryzae pv. oryzae 90, Xanthomonas oryzae pv. oryzae 599, Xanthomonas oryzae pv. oryzae 710, Janibacter melonis, Ralstonia solanacearum race 1, Ralstonia solanacearum race 3, Candida glabrata, Candida krusei, Candida tropicalis, Saccharomyces cerevisiae 5312, Burkholderia glumae SL 2870, Burkholderia glumae SL 2399, or Burkholderia glumae R1, but is not limited thereto.

The present invention furthermore provides a method for increasing resistance to plant pathogen by inducing an immune reaction of a plant wherein said method comprises a step of treating the plant with bacterial genetic materials. Preferably, said bacterial genetic material is bacterial RNA.

Mentioned bacteria include the following gram-negative bacteria; Haemophilus influenzae, Yersinia pestis, Escherichia coli, Salmonella typhi, Salmonella typhimurium, Vibrio cholerae, Pseudomonas aeruginosa, and Pseudomonas syringae, but are not limited thereto, as well as the following gram-positive bacteria; genus Bacillus consisting of Bacillus stearothermophilus, Bacillus cereus, and Bacillus anthrasis, Paenibacillus polymyxa, Clavibacter michiganensis, and Pectobacterium carotovorum, but are not limited thereto.

In addition to said bacterial RNA, a plant can be further treated with BTH (2,1,3-benzothiadiazole), ethephon, salicylic acid, methyl jasmonate, or DL-β-amino-n-butyric acid. For a method of treating a plant with bacterial genetic materials, any method publicly known in the pertinent art, for example, spray and irrigation, etc. can be employed.

According to a method of one embodiment of the present invention, the above-mentioned plant pathogen can be selected from a group consisting of gram-positive bacteria, gram-negative bacteria and fungi. Preferably, mentioned plant pathogen can be Clavibacter michiganensis subsp. Michiganensis, Pseudomonas syringae pv. tomato DC3000, Pectobacterium carotovorum subsp. Carotovorum, Xanthomonas campestris pv. vesicatoria 833, Xanthomonas campestris pv. vesicatoria 833 pila, Pectobacterium carotovorum subsp. Atrosepticum, Acidovorax konjaci, Xanthomonas albilineans, Xanthomonas oryzae pv. oryzae 90, Xanthomonas oryzae pv. oryzae 599, Xanthomonas oryzae pv. oryzae 710, Janibacter melonis, Ralstonia solanacearum race 1, Ralstonia solanacearum race 3, Candida glabrata, Candida krusei, Candida tropicalis, Saccharomyces cerevisiae 5312, Burkholderia glumae SL 2870, Burkholderia glumae SL 2399, or Burkholderia glumae R1, but is not limited thereto.

The present invention still further provides a plant produced by said method to have increased resistance to plant pathogen.

Mentioned plant can be food crops that are selected from a group consisting of rice, wheat, barley, corn, soy bean, potato, red bean, oat and millet; vegetable crops that are selected from a group consisting of Arabidopsis thaliana, Chinese cabbage, radish, hot pepper, strawberry, tomato, watermelon, cucumber, cabbage, melon, zucchini, scallion, onion and carrot; special crops that are selected from a group consisting of ginseng, tobacco, cotton, sesame, sugar cane, sugar beet, wild sesame, peanut and rapseed; fruits that are selected from a group consisting of apple, pear, date, peach, kiwi, grape, tangerine, orange, persimmon, plum, apricot and banana; flowers that are selected from a group consisting of rose, gladiolus, gerbera, carnation, chrysanthemum, lily, and tulip; and feed crops that are selected from a group consisting of rye grass, red clover, orchard grass, alfalfa, tall fescue, and perennial rye grass. Preferably, mentioned plant can be a dicotyledonous plant including Arabidopsis thaliana, egg plant, tobacco, hot pepper, tomato, burdock, crown daisy, lettuce, Chinese bellflower, spinach, chard, yam, celery, carrot, dropwort, parsley, Chinese cabbage, cabbage, leaf radish, watermelon, melon, cucumber, zucchini, gourd, strawberry, soy bean, mung bean, kidney bean and green pea, etc. Still more preferably, said plant is Arabidopsis thaliana.

The present invention still further provides seeds of the above-described plant having resistance to plant pathogen. Preferably, said seeds are the seeds of Arabidopsis thaliana.

The present invention will now be described in greater detail with reference to the following examples. However, it is only to specifically exemplify the present invention and in no case the scope of the present invention is limited by these examples.

### EXAMPLES

### Example 1

### Resistance to Plant Disease is Increased by Bacterial RNA

In order to observe an effect of increasing resistance to disease by innate immunity of plants that is induced by bacterial RNA, Arabidopsis thaliana Col-0 and Pseudomonas syringae pv. tomato DC3000 were first selected as a test plant and a plant pathogen, respectively. For a positive control, BTH (2,1,3-benzothiadiazole), which is known to be capable of inducing resistance in plant, was used. Water was used as a solvent for RNA and BTH, as well as a negative control for the test.

1-1) Isolation of Bacterial RNA

P. syringae pv. tomato DC3000 was cultured in King's B liquid medium (0.15% K₂HPO₄, 0.15% MgSO₄.7H₂O, 2% proteose peptone No. 3; Difco), while Paenibacillus polymyxa E681 was cultured in Trypic soy broth (17 g Pancreatic digest of casein, 3 g Enzymatic digest of soybean meal, 5 g dextrose, 2.5 g sodium chloride; BD, Becton, Dickinson and Company, LOT no. 7079938) until its OD₆₀₀ reached two. Then, total RNA was isolated and purified therefrom using RNeasy plus mini kit (Qiagen). During such process, bacterial DNA and proteins were removed (DNA contamination was again checked by using electrophoresis).

1-2) Injection of Bacterial RNA and Infiltration of Pathogen

In order to observe plant's resistance to disease in the treatment of Arabidopsis thaliana with bacterial RNA, the bacterial RNA was first diluted to a concentration of 150 ng/μl and then injected to the backside of the leaves of Arabidopsis thaliana, which had been grown in soil for two weeks. For a positive control, 0.33 mM BTH solution was also injected with the same manner as described for the bacterial RNA. Five days after the RNA treatment, a plant pathogen P. syringae pv. tomato DC3000 was cultured in King's B broth for one day, followed by being diluted to a concentration of 10⁵cfu/mL, and then infiltrated into three spots except the leaves treated with the RNA.

1-3) Observation of a Symptom of Disease

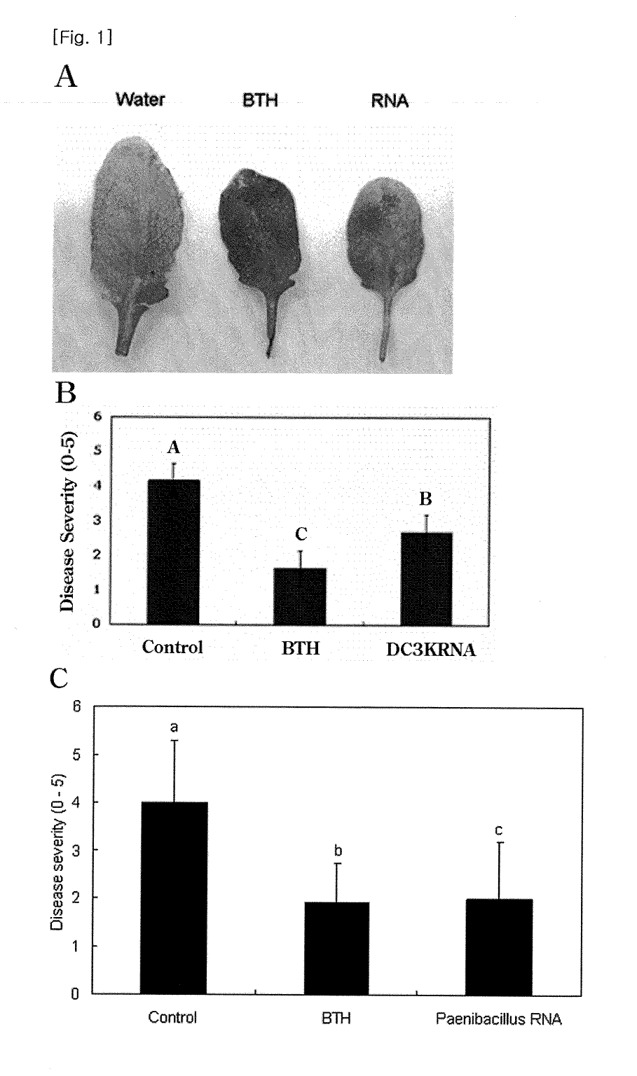

Five days after the injection of P. syringae pv. tomato DC3000, plant's symptom of disease was observed and then statistically analyzed by recording it with a number from 0 to 5 scale which corresponds to severity of the disease. FIG. 1 shows resistance to plant diseases wherein the resistance of the plant was induced by treating the plant with bacterial RNA. Specifically, panel A shows a photographic image of a plant leaf which was treated with water (negative control), BTH (positive control), or pre-treated with the bacterial RNA and then treated with P. syringae pv. tomato DC3000, wherein the photographic image was taken five days after the treatment. Panel B shows the disease controlling effect of RNA from gram-negative Pseudomonas syringae pv. tomato strain DC3000, compared to others. Panel C shows the disease controlling effect of RNA from gram-positive Paenibacillus polymyxa strain E681, compared to others.

As it can be seen from FIG. 1, although it is not as strong as the group treated with BTH, the group treated with the RNA from gram-negative bacteria showed increased resistance to plant disease compared to the water treatment group. In addition, it was found that the group treated with the RNA from gram-positive bacteria showed resistance to plant disease almost the same as the group treated with BTH.

### Example 2

### Determination of Gene Expression Profile of the Plant in Accordance with the Treatment with Bacterial RNA

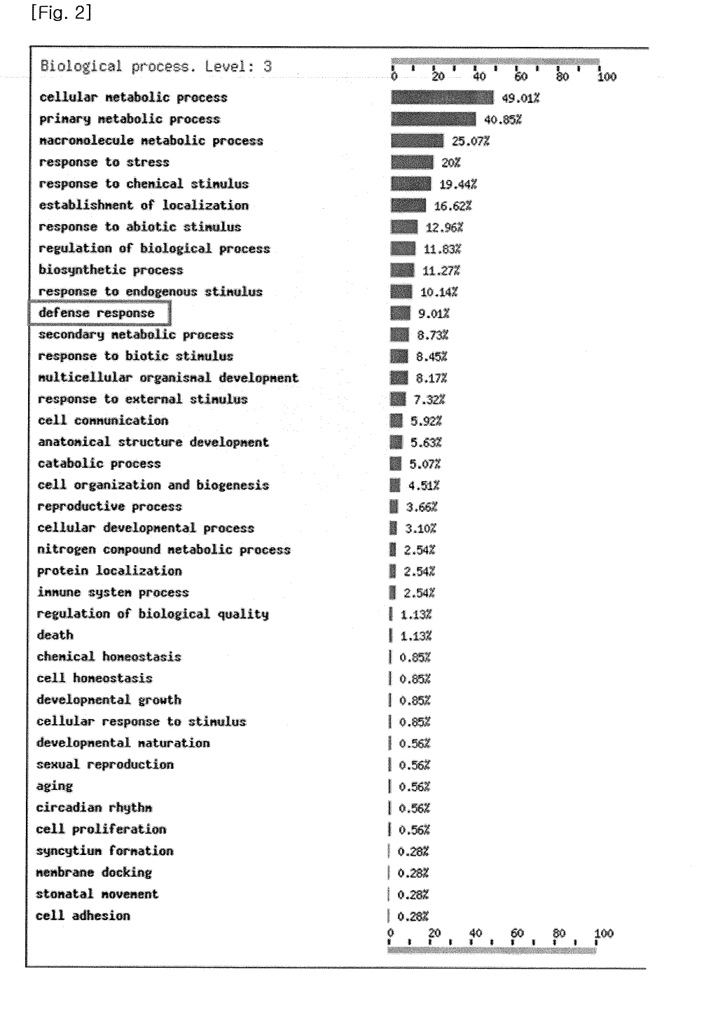

It was confirmed above that, when a plant was pre-treated with bacterial RNA and then infected with a plant pathogen, resistance to the pathogen is improved compared to those without such pre-treatment. In order to find out the gene expression profile of the plant in such case, a microarray analysis was carried out for Arabidopsis thaliana (Affimatrix 50K Arabidopsis microarray). Specifically, after treating a plant sample with RNA or water, respectively, for six hours, total RNA was isolated from the plant and purified for the comparison. FIG. 2 shows a gene expression profile and an analysis of gene ontology of the plant, upon its treatment with bacterial RNA. As a result of carrying out gene ontology analysis on biological processes of a gene cluster of which expression level had been up-regulated with the treatment of the bacterial RNA, it was confirmed that expression level was increased for many kinds of genes. It was further confirmed that various genes that have been known to be related with resistance to disease or response to stress in plant were included among said many kinds of genes. In the following Table 1, genes that have been up-regulated with the treatment of the bacterial RNA, as known from the result of Affimatrix 50K Arabidopsis microarray assay, are listed.

TABLE 1

| Fold Change | Gene Symbol | Gene Title | InterPro |
|---|---|---|---|
| 8.70199 | RAP2.6 | RAP2.6; DNA binding/ | Pathogenesis-related transcriptional |
| | | transcription factor | factor and ERF |
| 7.926122 | ATBETA-AMY | ATBETA-AMY (BETA-AMYLASE); | Glycoside hydrolase, family 14B, plant; |
| | | beta-amylase | Glycoside hydrolase, family 14 |
| 7.8291006 | RNS1 | RNS1 (RIBONUCLEASE 1); | Ribonuclease T2 |
| | | endoribonuclease | |
| 7.544235 | AT2G43620 | chitin binding/chitinase | Chitin-binding, type 1; |
| | | | Glycoside hydrolase, family 19 |
| 6.9990225 | PEARLI 1 1 | PEARLI 1 1; lipid binding | Plant lipid transfer/seed storage/ |
| | | | trypsin-alpha amylase inhibitor |
| 6.9194713 | AT1G51850 | kinase | Leucine-rich repeat; Protein kinase |
| 6.3413553 | | leucine-rich repeat family | Leucine-rich repeat |
| | | protein | |
| 6.074326 | AT3G11340 | UDP-glycosyltransferase/transferase, | UDP-glucoronosyl/UDP-glucosyl transferase |
| | | transferring hexosyl groups | |
| 5.8225365 | LTP4 | LTP4 (LIPID TRANSFER PROTEIN 4); | Plant lipid transfer protein/Par alLergen; |
| | | lipid binding | Plant lipid transfer/seed storage/ |
| | | | trypsin-alpha amylase inhibitor |
| 5.5968485 | SRG1 | SRG1 (SENESCENCE-RELATED GENE 1) | 2OG-Fe(II) oxygenase superfamily |
| 5.5915146 | AT3G46660 | UDP-glycosyltransferase/tansferase, | UDP-glucorOnosyl/ |
| | | transferring glycosyl groups | UDP-glucosyl transferase |
| 5.574066 | AT1G10585 | transcription factor | |
| 5.5394964 | AT2G45210 | unknown protein | Auxin responsive SAUR protein |
| 5.5000825 | AT1G30700 | electron carrier | FAD linked oxidase, N-terminal |
| 5.4191217 | AT4G12500 | lipid binding | Plant lipid transfer/seed storage/ |
| | | | trypsin-alpha amylase inhibitor |
| 5.4170103 | AT2G38240 | unknown protein | 2OG-Fe(II) oxygenase superfamily |
| 5.1754513 | DIN2 | DIN2 (DARK INDUCIBLE 2); hydrolase, | Glycoside hydrolase, family 1 |
| | | hydrolyzing O-glycosyl compounds | |
| 5.147278 | AT4G22470 | lipid binding | Plant lipid transfer/seed storage/ |
| | | | trypsin-alpha amylase inhibitor |
| 5.132769 | AT1G73260 | endopeptidase inhibitor | Trypsin inhibitor Kunitz |
| 5.1232004 | AT5G07010 | sulfotransferase | Sulfotransferase |

1. A composition for increasing resistance to plant pathogen by inducing an immune reaction of a plant wherein said composition comprises bacterial genetic materials as an effective component.

2. The composition according to claim 1, characterized in that said bacterial genetic material is bacterial RNA.

3. The composition according to claim 1, characterized in that it further comprises BTH (2,1,3-benzothiadiazole), ethephon, salicylic acid, methyl jasmonate, or DL-β-amino-n-butyric acid.

4. The composition according to claim 1, characterized in that said plant pathogen is selected from the group consisting of gram-positive bacteria, gram-negative bacteria and fungi.

5. The composition according to claim 4, characterized in that said plant pathogen is selected from the group consisting of Clavibacter michiganensis subsp. Michiganensis, Pseudomonas syringae pv. tomato DC3000, Pectobacterium carotovorum subsp. Carotovorum, Xanthomonas campestris pv. vesicatoria 833, Xanthomonas campestris pv. vesicatoria 833 pila, Pectobacterium carotovorum subsp. Atrosepticum, Acidovorax konjaci, Xanthomonas albilineans, Xanthomonas oryzae pv. oryzae 90, Xanthomonas oryzae pv. oryzae 599, Xanthomonas oryzae pv. oryzae 710, Janibacter melonis, Ralstonia solanacearum race 1, Ralstonia solanacearum race 3, Candida glabrata, Candida krusei, Candida tropicalis, Saccharomyces cerevisiae 5312, Burkholderia glumae SL 2870, Burkholderia glumae SL 2399, and Burkholderia glumae R1.

6. A method for increasing resistance to plant pathogen by inducing an immune reaction of a plant wherein said method comprises a step of treating the plant with bacterial genetic materials.

7. The method according to claim 6, characterized in that said bacterial genetic material is bacterial RNA.

8. The method according to claim 6, characterized in that the plant is further treated with BTH (2,1,3-benzothiadiazole), ethephon, salicylic acid, methyl jasmonate, or DL-β-amino-n-butyric acid.

9. The method according to claim 6, characterized in that said plant pathogen is selected from the group consisting of gram-positive bacteria, gram-negative bacteria and fungi.

10. The method according to claim 9, characterized in that said plant pathogen is selected from the group consisting of Clavibacter michiganensis subsp. Michiganensis, Pseudomonas syringae pv. tomato DC3000, Pectobacterium carotovorum subsp. Carotovorum, Xanthomonas campestris pv. vesicatoria 833, Xanthomonas campestris pv. vesicatoria 833 pila, Pectobacterium carotovorum subsp. Atrosepticum, Acidovorax konjaci, Xanthomonas albilineans, Xanthomonas oryzae pv. oryzae 90, Xanthomonas oryzae pv. oryzae 599, Xanthomonas oryzae pv. oryzae 710, Janibacter melonis, Ralstonia solanacearum race 1, Ralstonia solanacearum race 3, Candida glabrata, Candida krusei, Candida tropicalis, Saccharomyces cerevisiae 5312, Burkholderia glumae SL 2870, Burkholderia glumae SL 2399, and Burkholderia glumae R1.

11. A plant produced by a method described in claim 6 to have increased resistance to plant pathogen.

12. The plant according to claim 11, characterized in that it is a dicotyledonous plant.

13. Seeds of the plant according to claim 11.

14. A plant produced by a method described in claim 7 to have increased resistance to plant pathogen.

15. A plant produced by a method described in claim 8 to have increased resistance to plant pathogen.

16. A plant produced by a method described in claim 9 to have increased resistance to plant pathogen.

17. A plant produced by a method described in claim 10 to have increased resistance to plant pathogen.
